(19) United States
(12) Patent Application Publication
(10) Pub. No.: US 2019/0350605 A1
(43) Pub. Date: Nov. 21, 2019

(54) SUCTION FORCEPS FOR ENDOSCOPIC SURGERY

(71) Applicants: Nobuyuki SAKURAZAWA, Tokyo (JP); HIRATA PRECISIONS CO., LTD., Ichikawa-shi, Chiba (JP)

(72) Inventor: Nobuyuki SAKURAZAWA, Tokyo (JP)

(21) Appl. No.: 16/461,468

(22) PCT Filed: Jun. 16, 2017
(86) PCT No.: PCT/JP2017/022277

§ 371 Date: May 16, 2019

(30) Foreign Application Priority Data
Nov. 18, 2016 (JP) PCT/JP2016/084357

(51) Int. Cl. A61B 17/22; A61B 17/29; A61B 1/06; A61B 18/14

(52) U.S. Cl. CPC A61B 17/22031; A61B 17/29; A61B 1/0676; A61B 2018/00589; A61B 2217/005; A61B 2017/2947; A61B 2017/22079; A61B 18/1445

(57) ABSTRACT

To provide suction forceps for endoscopic surgery which are capable of quickly aspirating a fluid such as blood or another body fluid without requiring reinsertion of a dedicated aspiration tube even when there is leakage of blood or another body fluid during surgery, and consequently, also make it possible to prevent the duration of surgery from lengthening. Thus, suction forceps for endoscopic surgery are provided which are equipped with: a hollow section formed in a body section; a gripping part which manages tissue during surgery, and is formed at the tip end of the body section on one side thereof; and a suction port which is formed so as to be connected to the hollow section, and aspirates body fluid that has leaked from the tissue or blood that was effused during the surgery.

### FIELD OF THE INVENTION

This invention relates to a suction forceps for endoscopic surgery having a suction function employed during surgery in use of an endoscope.

### BACKGROUND OF RELATED ART

In endoscopic surgery using such as, e.g., laparoscope, and thoracoscope, conventionally, a scope, a forceps, an electric scalpel, and the like are inserted through a port (trocar) attached to such as, e.g., an abdominal wall or a chest wall, for surgical treatments such as, excision, hemostasis, suture in the coelom.

For example, forceps of various types, having a gripping part at a tip of the tool for gripping targeted tissues or suture needles, have been known (see, e.g., Patent Document No. 1), but no forceps has been known in which a suction port is formed in the gripping part for suctioning liquid such as blood effused and body liquid leaking out of the tissues during surgery.

### PRIOR ART DOCUMENTS

Patent Document No. 1: Japanese Patent Application Publication (A1) 2010-36,024

### SUMMARY OF THE INVENTION

When blood is effused during surgery, it was previously the only way to temporarily pull out the forceps from the port and to suck the effused blood after reinserting a special suction pipe. Accordingly, the surgical field may not be ensured until insertion of the suction pipe and suction of the blood, therefore raising a problem to cause the bleeding amount larger resultantly and to make the operation period longer, because bleeding may not be suppressed until completion of a treatment stopping bleeding.

This invention is made in consideration of those actual situations. It is an object of the invention to provide a suction forceps for endoscopic surgery capable of rapidly suctioning liquids such as blood and body liquid in not requiring any re-insertion of a special suction pipe even where it is bleeding or leaking body liquid during surgery, thereby preventing a surgical period from becoming longer.

### Means for Solving the Problems

To solve the above problems, the suction forceps for endoscopic surgery according to the invention includes a hollow section formed in a body section, a gripping part formed on a tip of the body section on one end side of the body section for handling tissues during surgery, and a suction port formed in communication with the hollow section for suctioning effused blood and body liquid leaking from the tissues during the surgery.

It is preferable to render the gripping part have an annular portion fitting to an inclined end surface portion formed in an oval shape at the tip of the body section on one end side of the body section and function as a blood clot crushing member for crushing the blood clot by clamping the blood clot with the annular portion and the inclined end surface portion.

The inclined end surface portion may be formed with an end surface side opening for forming the suction port, and the annular portion fitting the inclined end surface portion may be formed with a crashing opening having a diameter smaller than that of the end surface side opening.

Furthermore, a reservoir section may be formed in communication with the hollow section at the other end side of the body section having the gripping part formed for reserving the blood and the body liquid suctioned through the suction port.

The reservoir section may have a body liquid absorbing material for absorbing the blood and the body liquid.

The reservoir section may have a connection pipe detachably attached to a suction pipe extending from an external suction device.

A switching means for turning on and off suction operation using the external suction device is preferably provided, while connected to the suction pipe from the external suction device via the connection pipe.

The suction forceps for endoscopic surgery according to the invention may include an illuminating means for externally illuminating light via the sucking port.

The suction forceps for endoscopic surgery according to an embodiment may include a connection terminal connecting a conduction line extending from an external electric coagulation apparatus, and the suction forceps may coagulate in a carbonizing manner a part at which bleeding is to be stopped based on a power generated at the external electric coagulation apparatus.

The connection terminal at that time may be connected to the gripping part and be formed at an end of a manipulation wiring on an end side of thereof for manipulating the gripping part.

The suction forceps for endoscopic surgery according to the invention may have a suction stopping mechanism for stopping suction operation done at the suction port.

In this situation, the suction stopping mechanism is preferably arranged at the gripping part, and the gripping part may include a gripping piece portion arranged pivotally to the body section for performing suction and stopping suction at the suction port in accordance with pivotal movement of the gripping piece portion with respect to the body section.

It is to be noted that the suction port for performing suction and stopping suction in accordance with opening and closing movements of the gripping piece portion with respect to the body section, may be formed at any of the tip of the body section on one end side of the body section, the side surface of the body section, and the bottom surface of the body section.

### Advantages of the Invention

According to the invention, a suction forceps for endoscopic surgery can be provided in being capable of rapidly suctioning liquids such as blood and body liquid in not requiring any re-insertion of a special suction pipe even where it is bleeding or leaking body liquid during surgery, thereby preventing a surgical period from becoming longer.

### BRIEF DESCRIPTION OF DRAWINGS

FIG. 1 is a schematic cross section showing a whole structure of a suction forceps 100 for endoscopic surgery according to a first embodiment;

FIG. 2 is a diagram showing suction operation in use of the suction forceps 100 for endoscopic surgery;

FIG. 3 is a partly enlarged view showing a structure of a suction port 104 formed on a one end side of the suction forceps 100 for endoscopic surgery;

FIG. 4 is illustration showing operation of a blood clot crushing member 105 according to the first embodiment;

FIG. 5 is illustration showing mist existing in a body cavity and mist of a mixture of blood;

FIG. 6 is a diagram showing suction operation for mist existing in a body cavity and mist of a mixture of blood;

FIG. 7 is a schematic view showing an illumination unit 116 provided at the suction forceps 100 for endoscopic surgery according to the first embodiment;

FIG. 8 is illustration showing operation of carbonized coagulation of a hemostasis point on a basis of electric power generated at an external electric coagulation apparatus 400;

FIG. 9 is a schematic view showing a situation when the suction forceps 100 for endoscopic surgery according to the first embodiment is actually used for an endoscopic surgery;

FIG. 10 is illustration showing a structure on a tip side of a body section of a suction forceps 700 for endoscopic surgery according to a second embodiment;

FIG. 11 is illustration showing a suction stopping mechanism 800 according to the second embodiment;

FIG. 12 is illustration showing operation states of the suction stopping mechanism 800 according to the second embodiment;

FIG. 13 is illustration showing another structure on the tip side of the body section of a suction forceps for endoscopic surgery according to the second embodiment;

FIG. 14 is illustration showing yet another structure on the tip side of the body section of a suction forceps for endoscopic surgery according to the second embodiment; and

FIG. 15 is illustration showing a conventional suction pipe structure.

### DESCRIPTION OF EMBODIMENTS

Hereinafter, referring to the drawings, embodiments according to the invention are described. It is to be noted that the invention is not limited to the following descriptions, and can be modified as far as not deviated from the subject matter of the invention.

### First Embodiment

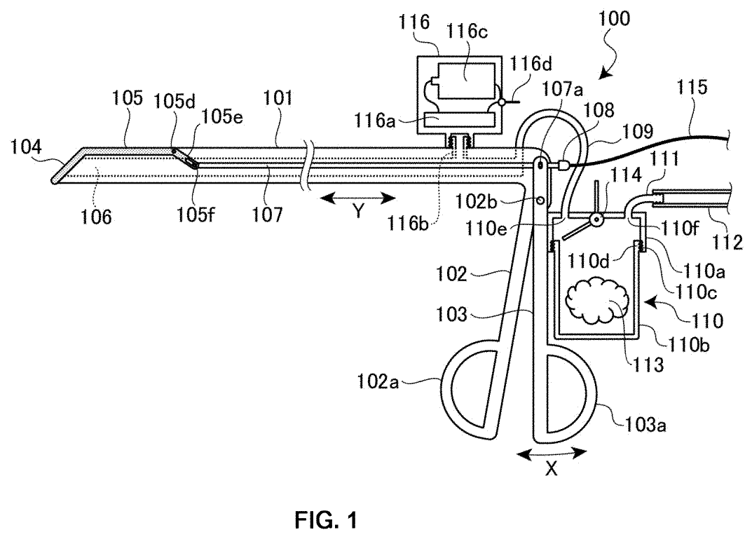

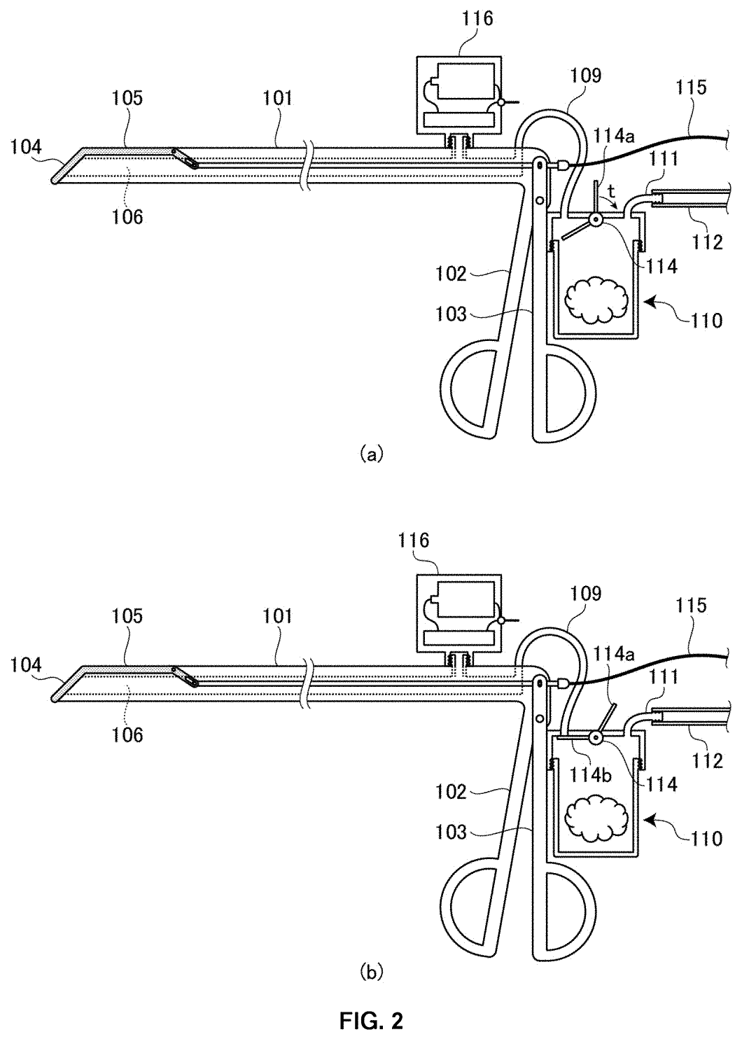

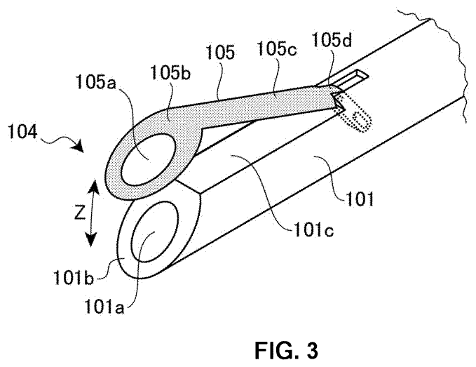

FIG. 1 is a schematic cross section showing a whole structure of a suction forceps 100 for endoscopic surgery according to this embodiment; FIG. 2 is a diagram showing suction operation in use of the suction forceps 100 for endoscopic surgery; FIG. 3 is a partly enlarged view showing a structure of a suction port 104 formed on a one end side of the suction forceps 100 for endoscopic surgery.

As shown in FIG. 1, the suction forceps 100 for endoscopic surgery according to this embodiment includes a body section 101 formed in a rod shape having a hollow section 106, a first control handle section 102 formed to extend in substantially a vertical direction from an end of the body section 101 in a lengthwise direction with a finger insertion portion 102a to insert an index finger of a surgeon, a second control handle section 103 arranged pivotally around a pivot portion 102b formed at the first control handle section 102 with a finger insertion portion 103a to insert a thumb of the surgeon, a suction port 104 formed on the other end, or a tip, of the body section 101 in the lengthwise direction formed with the first control handle section 102 for suctioning liquids such as effused blood and body liquid leaking from the tissues, and a manipulation wire 107 transmitting manipulating transmittable force of the second control handle section 103 to a blood clot crushing member 105, as described below, for structuring the suction port 104.

The second control handle section 103 is structured to be pivotally movable in an arrow X-direction in FIG. 1 with respect to the first control handle 102 around the pivot portion 102b as a center. The manipulation wire 107 is connected via a connector 107a to the tip of the second control handle 103, and when the manipulating wiring 107 drives in the arrow Y-direction in FIG. 1 as pivotal moving of the second control handle section 103 with respect to the first control handle section 102, a crushing body section 105c connected via a crushing connection portion 105f as described below can be made open and closed in Z-direction in FIG. 3.

A reservoir section 110 is formed at a top portion of the second control handle section 103 for reserving liquids such as blood and body liquids suctioned through the suction port 104 and the hollow section 106. The reservoir section 110 has a reservoir lid 110a directly secured to the top portion of the second control handle section 103, and a reservoir body section 110b secured via a connection between an engaging portion 110c formed on an inner wall of the reservoir lid 110a and an engaged portion 110d. The reservoir body section 110b is structured to be detachably attached to the reservoir lid 110a.

At a top face portion of the reservoir lid 110a, formed respectively are an opening 110e on a hollow side in communication with a connection pipe 109 on a hollow side formed continuously from the hollow section 106, and an opening 110f on a suction pipe side in communication with a connection pipe 111 connected to a suction pipe extending from a suction device described below. An opening and closing switch 114 is arranged at the top face portion between the opening 110e on the hollow side and the opening 110f on the suction pipe side for serving as switching means making open and closed the opening 110e on the hollow side. In a situation shown in FIG. 2(a), the opening and closing switch 114 is in a state of “open,” and therefore the suction line from the suction device becomes a connection state, so that suction is allowed by suction force from the suction device. On the other hand, where a first switching piece 114a is pushed in an arrow-t direction from a state shown in FIG. 2(a) to enter into a state of “closed” shown in FIG. 2(b), a second switch piece 114 seals the opening 110e on the hollow side to render the suction line from the suction device in the closed state, so that the suction by the suction force of the suction device can be stopped.

A liquid absorption material 113 such as a gauze is contained in a reserving space inside the reservoir body section 110b. With the liquid absorption material 113 inside the reservoir body section 110b, contamination in the reserving space can be minimized, and encroachment of the suction liquid to the suction device can be prevented. It is to be noted that because the reservoir body section 110b according to this embodiment is constituted to be detachably attached to the reservoir lid 110a, the liquid absorption 113 can be replaced easily.

Next, referring mainly to FIG. 1 and FIG. 3, the suction port 104 as a tip portion of the suction forceps for endoscopic surgery 100 in the lengthwise direction is described. As shown in FIG. 3, the suction port 104 includes a seat portion 101c having a flat surface portion extending parallel to a center shaft of the body section 101 from a prescribed position to an end surface of the body section 101 in a lengthwise direction, and an inclined end surface portion 101b formed in having an ellipse cross section from a vicinity of an end of the seat portion 101c to an outer peripheral surface of the body section 101 or namely, formed so that its whole shape is in a substantially diagonally cut columnar body when an appearance of the body section 101 is viewed. The inclined end surface portion 101b is formed with an opening 101a on an end surface side in communication with the hollow section 106, thereby bringing liquids such as effused blood and body liquids leaking out of the tissues through the opening 101a on the end surface side.

The suction port 104 is formed with the blood clot crushing member 105 serving as a gripping part for handling tissues, e.g., picking up tissues during the surgery, as well as for crushing blood clots, e.g., blood clots in a state of soft jelly as time lapse. The blood clot crushing member 105 has a crushing body section 105c opening and closing in Z-direction in FIG. 3 around a crushing pivot 105d as a pivotal center according to pivotal operation of the second control handle section 103 in having substantially the same area as the flat surface portion of the seat portion 101c. As shown in FIG. 1, a crushing pivotal moving piece 105e having a crushing connection portion 105f is formed as extending at one end of the crushing body section 105c. A long hole is provided in the crushing pivotal moving piece 105e so as to make the crushing connection portion 105f movable. With the structure of the crushing connection portion 105f movable to the long hole, according to pivotal movement of the second control handle section 103, or namely, according to drive of the manipulation wiring 107, the crushing body section 105c can be open and closed in Z-direction in FIG. 3 around the crushing pivot 105d as the pivotal center.

In returning back to FIG. 3, a crushing annular portion 105b is formed on the other end of the crushing body section 105c in a shape substantially the same as the inclined end surface portion 101b. A crushing opening 105a is formed in the crushing annular portion 105b so as to have a slightly smaller area than the opening 101a on the end surface side. By forming an area difference between the opening diameter of the opening 101a on the end surface side and the opening diameter of the crushing opening 105a, clung blood clots can be crushed and suctioned again according to opening and closing movements of the crushing body section 105c even where blood clots are clung to the suction port 104 during suction operation and the suction port 104 is clogged temporarily. As described above, the crushing annular portion 105b and the inclined end surface portion 101b of the blood clot crushing member 105 function as a gripping part capable of handling the tissues according to opening and closing operation of the crushing body section 105c when not used for crushing blood clots.

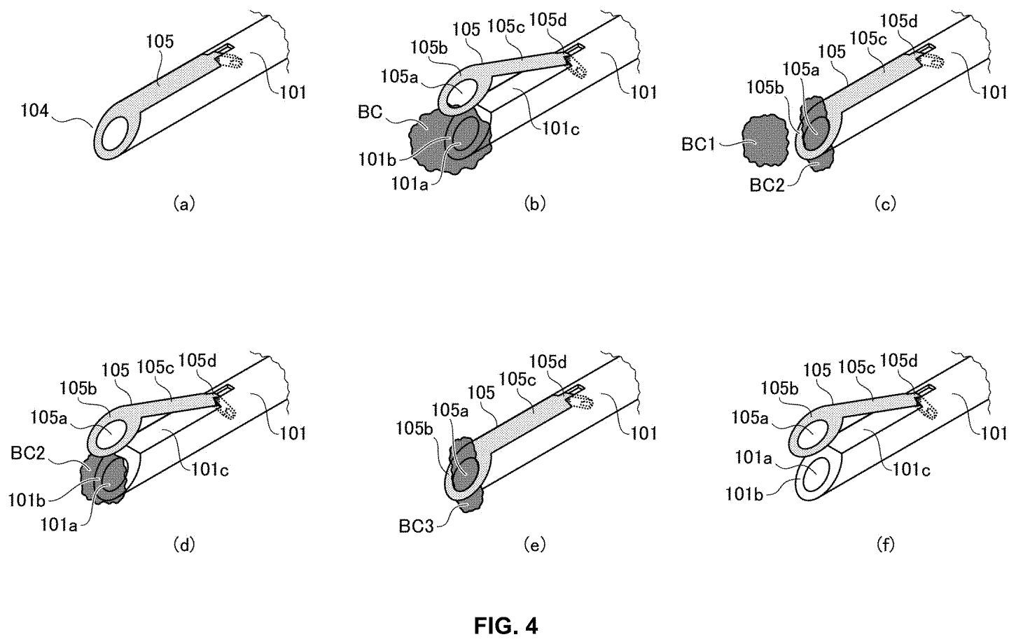

Blood clots crushing operation done with the blood clot crushing member 105 is described. In a suction state in which the crushing body section 105c is closed to the seat portion 101c, the blood clot BC is supposedly clung to clog the suction port 104, or the crushing opening 105a. From this state, the crushing body section 105c is made open and closed (FIG. 4(b)) as to clip the blood clot BC with the crushing annular portion 105b and the inclined end surface portion 101b. As shown in FIG. 4 (c), then, the blood clot BC is gradually divided into a blood clot BC1 and a blood clot BC2, and the blood clot BC2 is further divided into a blood clot BC3 (FIG. 4(d), and FIG. 4(e)); all of the blood clots BC are suctioned to return back the suction port 104 to the original state capable of suctioning as shown in FIG. 4(f).

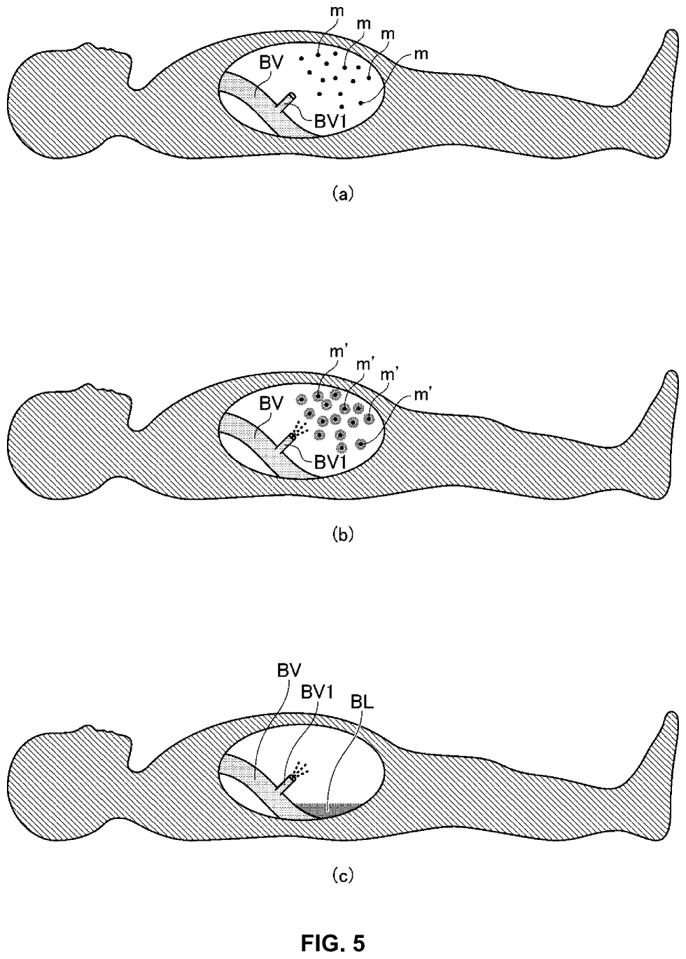

As shown in FIG. 5(a), mist m may occur from evaporation of the body liquid in a body cavity such as an abdominal cavity and a chest cavity. For example, as shown in FIG. 5(b), if bleeding occurs from blood vessels BV or fine blood vessels (blood capillaries) BV1 extending from blood vessels BV, mist comes into a state mixed with blood (m′). The suction forceps 100 for endoscopic surgery according to this embodiment may release the connection between the connection pipe 111 on the suction pipe side and the suction pipe from the suction device, and is capable of suctioning mist m existing in the body cavity and mist m′ mixed with blood even as used in a codeless state.

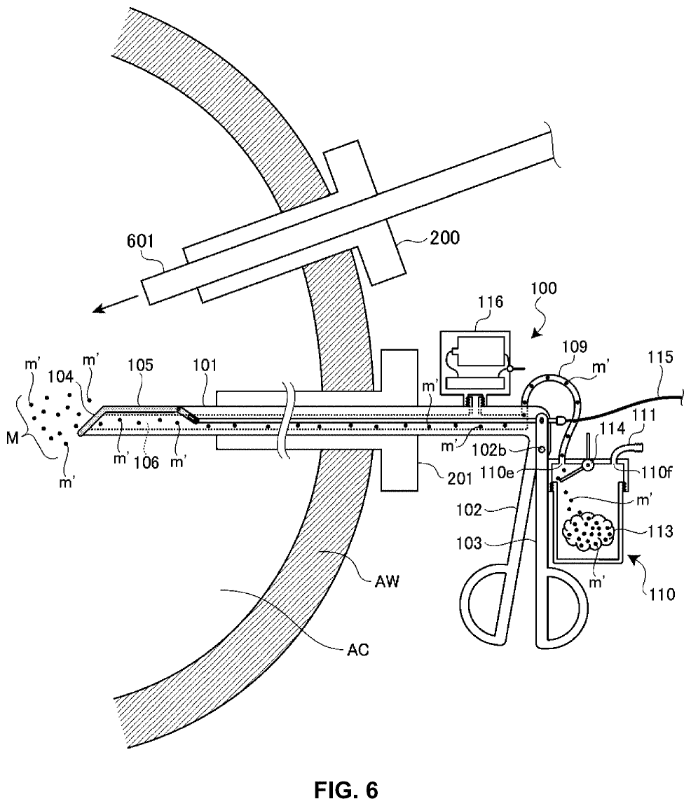

That is, in a typical endoscopic surgery, as shown in FIG. 6, carbon dioxide (CO₂) is continuously supplied to the interior of the body cavity AC with a pressure of +10 through 15 mmHg from a supply pipe 601 via a port 200 attached to a body wall AW from an insufflation device, the interior of the body cavity AC is maintained with the pressure of 760 mmHg plus 10 through 15 mmHg (intraabdominal pressure or intrachest pressure) where the pressure outside the body cavity is 760 mmHg (atmosphere). Accordingly, mist m and mist m′ mixed with blood can be exhausted from the pressure difference with respect to the exterior of the body cavity without any compulsory suction using the suction device merely by inserting the suction forceps 100 for endoscopic surgery according to this embodiment into the body cavity AC via the port 201. The exhausted mist m and the mist m′ mixed with blood are absorbed with the liquid absorbing material 113 such as a gauze provided in the reserving space inside the reservoir body section 110b, so that the suction forceps 100 can prevent the surgeon from directly exposing to those mists. Where the first switch piece 114a is set to the “closed” state, the pressure inside the body cavity can be maintained to a prescribed pressure. It is to be noted that as shown in FIG. 5(c), according to the suction forceps 100 for endoscopic surgery of this embodiment, blood BD gathered in the body cavity can be suctioned as a matter of course.

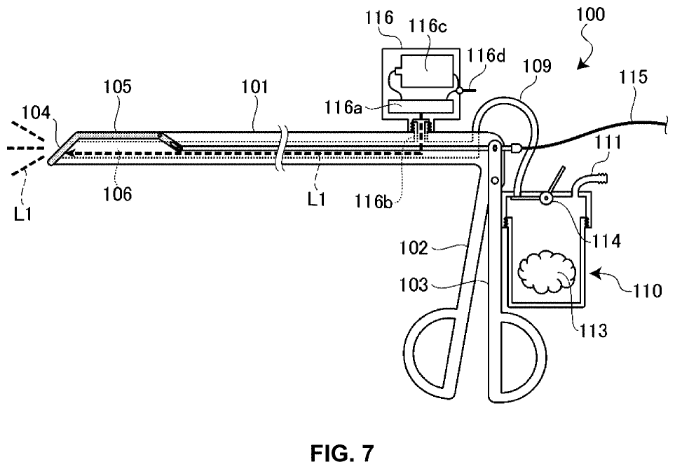

As shown in FIG. 1 and FIG. 7, the body section 101 of the suction forceps 100 for endoscopic surgery according to this embodiment has an illumination section 116. The illumination section 116 includes a light emitting element 116a such as an LED (light emitting diode), a light guidance section 116b having a converging lens for guiding light emitted from the light emitting element 116a to the hollow section 106, a power source section 116c having, e.g., a battery for supplying a drive electric power to the light emitting element 116a, and a switch 116d for turning on and off the light emission of the light emitting element 116a. As the body section 101 is thus formed with the illumination section 116, the surgical field can be illuminated brightly because light L1 can be emitted externally via the suction port 104 in a state that no liquid exists in the hollow section 106. It is to be noted that the illumination section 116 can be formed in a manner detachable from the body section 101 when necessary.

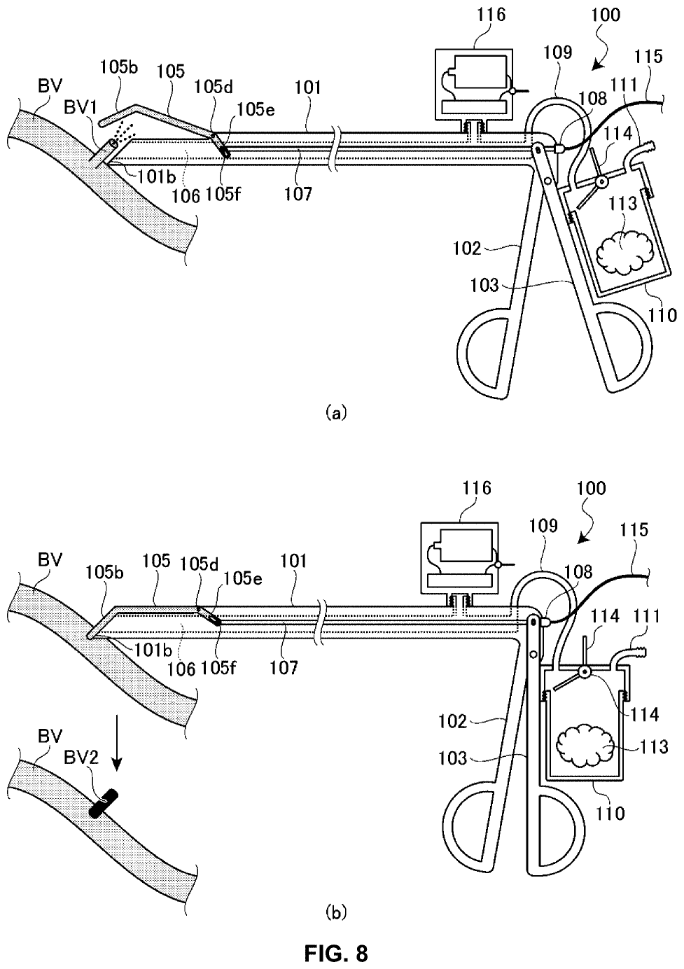

As shown in FIG. 8(a), the suction forceps 100 for endoscopic surgery according to this embodiment has a wiring connection terminal 108 serving as one end on a one side of the manipulation wire 107 connected to a power supply cord 115 extending from a main portion of an electric coagulation apparatus, not shown, thereby making areas, e.g., fine blood vessels BV1 and tissues, coagulated by providing power to coagulate the areas. That is, such as the manipulation wire 107, the blood clot crushing member 105, and the inclined end surface portion 101b are formed of a conducting material, and as shown in FIG. 8(b), power is supplied where the area to be coagulated (fine blood vessels BV1) is pinched with the crushing annular portion 105b and the inclined end surface portion 101b, thereby coagulating the area to be coagulated (fine blood vessels BV2) by carbonization.

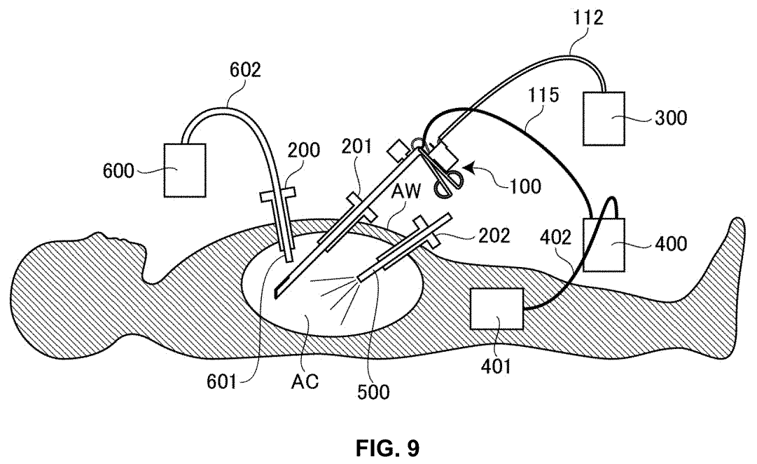

FIG. 9 is a schematic view showing a situation when the suction forceps 100 for endoscopic surgery according to the first embodiment is actually used for an endoscopic surgery.

The suction forceps 100 for endoscopic surgery is inserted into the body cavity AC via a port 201. Similarly, a supply pipe 601 connected to a ventilation line 602 of the insufflation device 600 via the port 200, and a laparoscope 500 via a port 202 are inserted into the body cavity AC, respectively.

During the surgery, as described above, the insufflation device 600 supplies carbon dioxide (CO₂) with a pressure of +10 through 15 mmHg. The surgeon performs the surgery as confirming the surgical field shown on an external display apparatus such as, e.g., a monitor, not shown, via the laparoscope 500.

The suction forceps 100 for endoscopic surgery is connected to a suction device 300 via the suction pipe 112. As described above, the suction forceps 100 for endoscopic surgery according to this embodiment is formed with the suction port 104 at the tip in the lengthwise direction for suctioning liquids such as effused blood and body liquids leaking out of the tissues. The suction port 104 is formed with the blood clot crushing member 105, and the blood clot crushing member 105 is structured to be capable of gripping and pinching tissues in substantially the same way as an ordinary forceps. According to the suction forceps 100 for endoscopic surgery of this embodiment, the suction forceps 100 can handle the tissues for the purpose of gripping the tissues, and at the same time can quickly absorb the liquids such as, e.g., blood and body liquids via the suction port 104 with suction force from the suction device 300 without any replacement of a particular suction pipe even when blood or body liquids are leaked from the blood vessels or the tissues.

Where the blood clot coagulated from the blood is clung to the suction port 104 and where the suction port 104 is closed, the blood clot can be crushed and divided into smaller ones and can be suctioned well according to the opening and closing operation of the crushing body section 105c.

The suction forceps 100 for endoscopic surgery of this embodiment can release the connection to the suction pipe 112 extending from the connection pipe 111 on the suction pipe side and the suction device 300, so that the suction forceps 100 can suction the mist m and the mist m′ mixed with blood existing in the body cavity based on the pressure difference between the inside and outside of the body cavity even where used in the cordless state.

As shown in FIG. 9, suction forceps 100 for endoscopic surgery is connected to the electric coagulation apparatus 400 via the power supply cord 115. It is to be noted that a counter electrode plate 401 is attached to the patient via a power supply cord 402.

By rendering the manipulation wire 107, the blood clot crushing member 105, and the inclined end surface portion 101b formed of a conductive material, the areas to be coagulated can be turned on with electricity while pinched with the crushing annular portion 105b and the inclined end surface portion 101b, thereby coagulating the areas to be coagulated by carbonization (fine blood vessels BV2).

As described above, according to the suction forceps 100 for endoscopic surgery of this embodiment, even where blood and body liquids are leaked during the surgery, the liquids such as blood and the body liquids can be quickly suctioned with no need of re-insertion of a particular suction pipe, and resultantly can prevent the surgical operation time from being extended.

### Second Embodiment

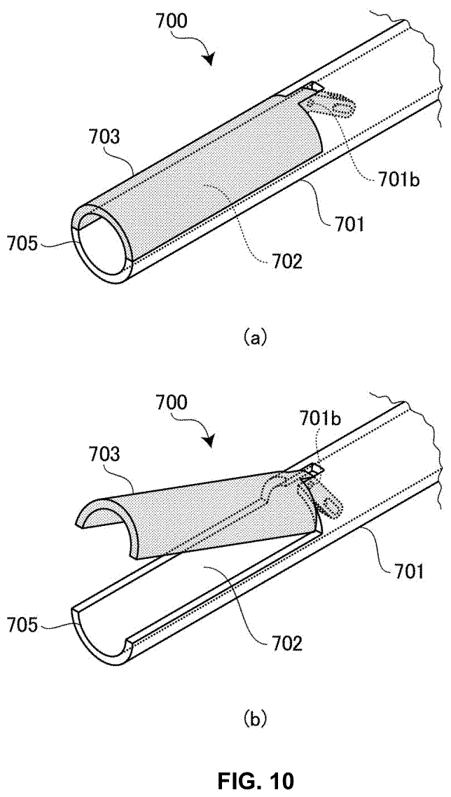

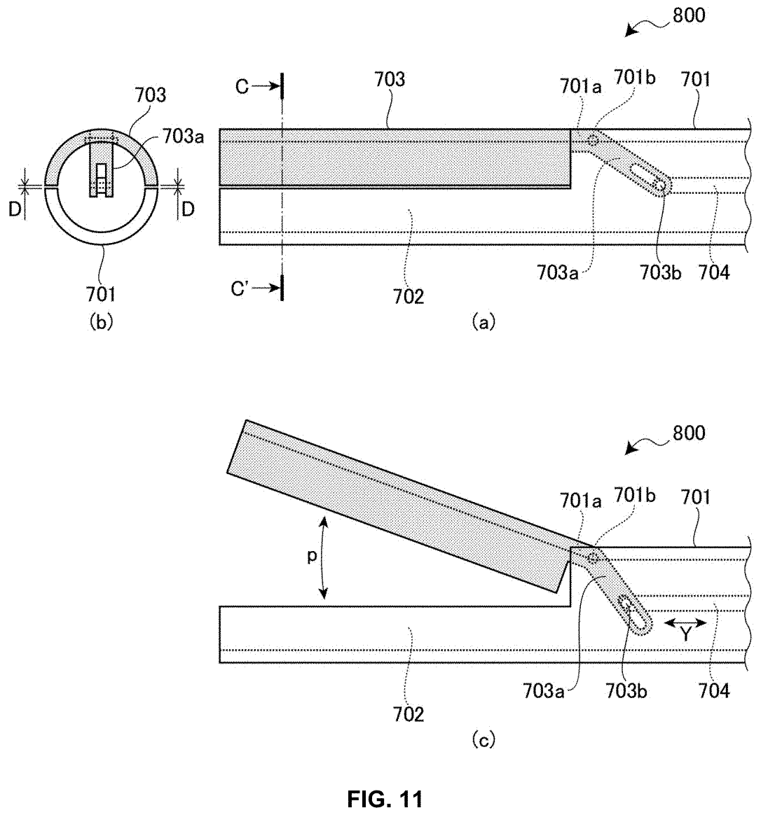

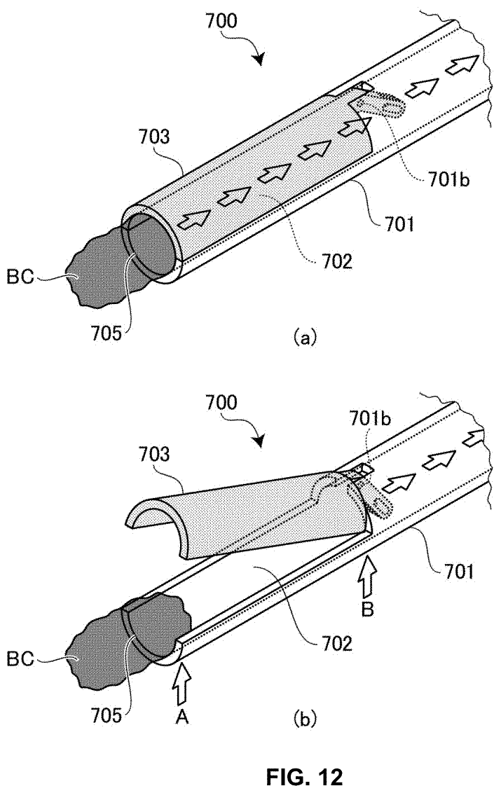

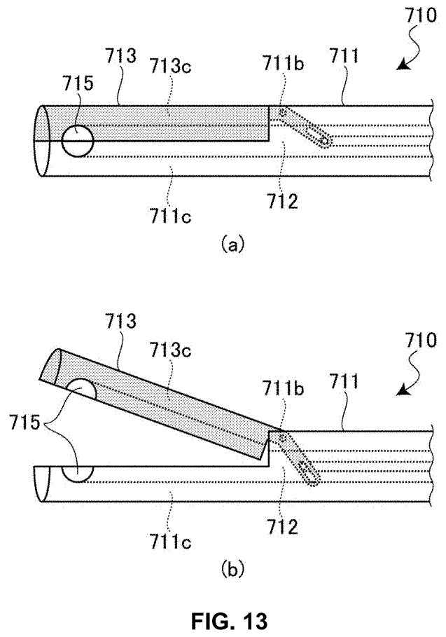

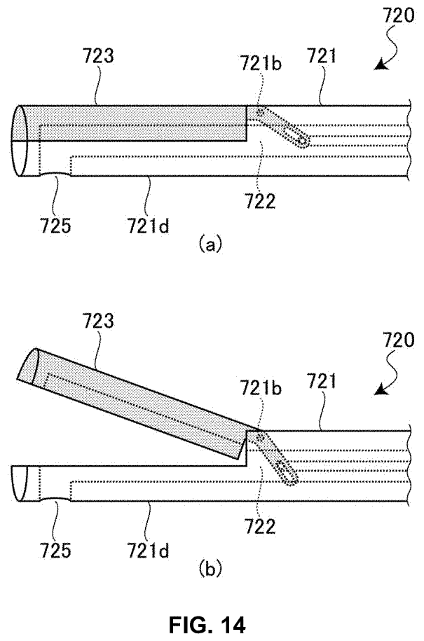

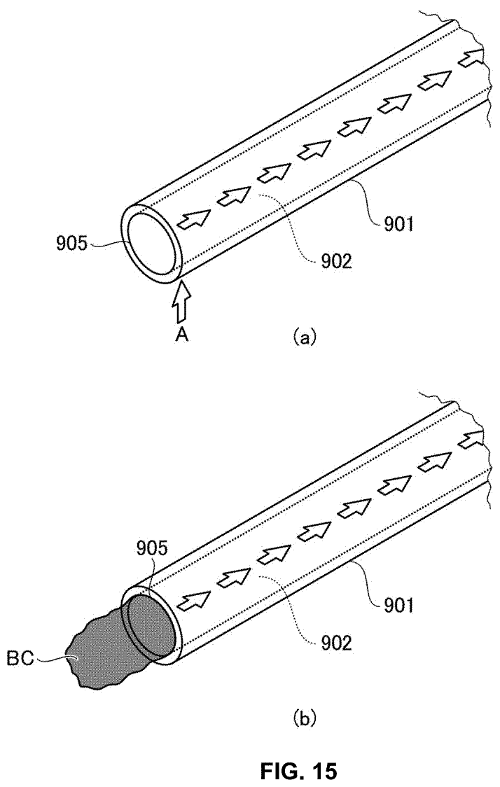

In a second embodiment, a structure having a suction stopping mechanism for stopping the suction at the suction port at the he suction forceps for endoscopic surgery of this invention, is described. FIG. 10 is illustration showing a structure on a tip side of a body section of a suction forceps 700 for endoscopic surgery according to a second embodiment; FIG. 11 is illustration showing a suction stopping mechanism 800 according to the second embodiment; FIG. 12 is illustration showing operation states of the suction stopping mechanism 800 according to the second embodiment; FIG. 13 is illustration showing another structure on the tip side of the body section of a suction forceps for endoscopic surgery according to the second embodiment; FIG. 14 is illustration showing yet another structure on the tip side of the body section of a suction forceps for endoscopic surgery according to the second embodiment. FIG. 15 is illustration showing a conventional suction pipe structure.

For example, as shown in FIG. 15, a suction pipe structure of a prior art type, which includes a body section 901, a hollow section 902, and a suction port 905, may exert suction pressure to a tip area A as the suction port 905 when liquids such as blood are suctioned.

If the blood is in a liquid state, the blood is not solidified at the tip area A as shown in FIG. 15(1), and is suctioned in an arrow direction in FIG. 15(1), but if solid substances such as blood clots in a jerry shape as blood coagulated are suctioned, the solid substances may be clogged at the tip area A (suction port 905) as shown in FIG. 15(2). In such a situation, the solid substances clogging to the suction port 905 are required to be removed upon pulling out the suction pipe externally one time, and it is inconvenient.

To the contrary, the suction forceps for endoscopic surgery according to this embodiment has a feature including a suction stopping mechanism capable of easily moving away solid substances from a suction port by stopping suction at the suction port even where the solid substances such as blood clots are suctioned. The following is a description of its structure.

FIG. 10 is illustration showing a structure on a tip side of a body section of a suction forceps 700 for endoscopic surgery according to a second embodiment; FIG. 10(1) is an illustration of a state (closed state) that a gripping piece 703 is closed with respect to a body section 701; FIG. 10(2) is an illustration of a state (open state) that the gripping piece 703 is made open with respect to the body section 701. It is to be noted that the structure of the first and second control handles manipulated by the surgeon, formed as extending in substantially a vertical direction from the opposite end of the body section in the lengthwise direction, may be formed in substantially the same way as in the first embodiment, and a description for those is omitted.

As shown in FIG. 10(1), the suction forceps 700 for endoscopic surgery has a hollow section 702, and has the gripping piece 703 formed pivotally with respect to the body section 701 around a pivot 701b as a pivot center, and a suction port 705 formed at a tip of the body section 701. The gripping piece 703 is structured to be formed in a half cylinder shape with a hollow so as to form a cylindrical hollow section 702 together with the body section 701 when in the closed state with respect to the body section 701 as shown in FIG. 10(1), 10(2).

FIG. 11 is illustration showing a suction stopping mechanism 800 in the suction forceps 700 for endoscopic surgery. FIG. 11(1) is an illustration of a structure in which the gripping piece 703 is in a closed state with respect to the body section 701; FIG. 11(2) is a cross section of C-C′ line; FIG. 11(3) is an illustration of a structure in which the gripping piece 703 is in an open state with respect to the body section 701. As shown in FIG. 11(1) to FIG. 11(3), the suction stopping mechanism 800 according to this embodiment includes the body section 701, the gripping piece 703, and the manipulation wire 704.

The gripping piece 703 is held as pivotally movable at a gripping piece support 701a structured as extending from the body section 701 around a pivot 701b as a pivot center, and as shown in FIG. 11(1) and FIG. 11(2), a manipulation wire connection portion 703a is provided as extending toward a center axis of the hollow section 702. The gripping piece 703 is connected with the manipulation wire 704 via a connection portion 703b at the manipulation wire connection portion 703a. As described in the first embodiment, the manipulation wire 704 according to the pivotal movement of the second control handle with respect to the first control handle is driven in arrow-Y direction in FIG. 11(3), the gripping piece 703 connected through the connection portion 703b and the manipulation wire connection portion 703a can move pivotally in p-direction in FIG. 11(3). The gripping piece 703 therefore can make transitions between double states, the open state that the gripping piece 703 is made open with respect to the body section and the closed state that made closed.

It is preferable to set a gap D between the body section and the gripping piece to be 0.15 mm or less as shown in FIG. 11(2) where the gripping piece 703 is in the closed state with respect to the body section 701. If the gap D is 0.15 mm or less, the gap is sealed by surface tension of liquid, so that suction may not be disturbed at the suction port 705.

Next, referring to FIG. 12, operation state of the suction stopping mechanism 800 is described. As shown in FIG. 12(1), if a blood clot BC as a solid substance is suctioned during a suction operation using the suction forceps 700 for endoscopic surgery having the suction stopping mechanism 800, the surgeon manipulates to move the second control handle pivotally with respect to the first control handle. According to drive of the manipulation wire 704 in accordance with the pivotal movement of the second control handle, the gripping piece 703 enters in the open state with respect to the body section 701 (FIG. 12(2)). Consequently, the suction pressure is shut down at the suction port 705 (tip area A), and a position exerting the suction pressure is shifted to a portion B as the pivotal movement position of the gripping piece 703. With this operation, suction to the blood clot BC at the suction port 705 is released, so that the blood clot BC can be moved away from the suction port 705 easily.

It is to be noted that in the suction operation during surgery, some weaken tissues in the living body may be inadvertently suctioned. In such a situation, there are risks of extermination of tissues and bleeding where the tissues clog in the suction port. In substantially the same way as the operation of mistakenly suctioning blood clots, according to the suction forceps 700 for endoscopic surgery having the suction stopping mechanism 800 of this embodiment, risks of extermination of tissues and bleeding can be reduced further because the tissues can be moved quickly easily away from the suction port 705 even where the weaken tissues are inadvertently suctioned.

In the description using FIG. 10 through FIG. 12, described are the structures having the suction port 705 at the tip portion (end) of the body section 701 as the suction forceps 700 for endoscopic surgery according to this embodiment. In this embodiment, arrangement positions of the suction port 705 at the body section 701 are not limited to the tip portion of the body section 701, and can be modified to various embodiments in consideration of factors such as, e.g., surgical methods, surgical environments, and habits of surgeons.

FIG. 13 is a diagram illustrating a structure of a suction forceps 710 for endoscopic surgery having a suction port 715 on a side surface of the body section 711. FIG. 13(1) is an illustration showing a structure that the gripping piece 713 is in a closed state with respect to the body section 711; FIG. 13(2) is an illustration showing a structure that the gripping piece 713 is in an open state with respect to the body section 711.

As shown in FIG. 13(1), the suction forceps 710 for endoscopic surgery includes a hollow section 712, and includes a gripping piece 713 formed pivotally with respect to the body section 711 around the pivot 711b as a pivot center, and a suction port 715 formed in side surface portions on a tip side of the body section 711. The suction port 715 is formed from each opening in a semicircular shape in a side surface portion 711c of the body section 711 and in a side surface portion 713c of the gripping piece 713, respectively, so as to form the suction port 715 in a circular shape in communication with the hollow section 712 when the gripping piece 713 is in the closed state with respect to the body section 711 as shown in FIG. 13(1), and FIG. 13(2).

With the embodiment shown in FIG. 13, exemplified is an example in which the suction port 715 is formed in the side surface portion on a near side when viewed from a diagram front side direction. With this embodiment, when the surgeon manipulates the suction forceps 710 for endoscopic surgery in holding the suction forceps 710 with his right hand, because the suction port 715 takes a position face to face over a scope to the surgeon, the surgeon can handle the tissues using the gripping section as well as can conduct suction operation in observing the suction port 715 during the surgery. The surgeon can operate more safely with this structure, and can further reduce risks of extermination of tissues and bleeding because blood clots and weaken tissues can be moved away from the suction port 715 quickly easily even where the blood clots and weaken tissues are inadvertently suctioned. It is to be noted that where the surgeon manipulates the suction forceps 710 for endoscopic surgery with his left hand, the suction port 715 is to be formed in a side surface portion on a rear side when viewed from a diagram front side direction, and the suction port may be provided in the side surface portion of each of the front and rear sides when viewed from a diagram front side direction.

FIG. 14 is diagram showing a structure of the suction forceps 720 for endoscopic surgery having a suction port 725 in a bottom surface of a body section 721. FIG. 14(1) is an illustration showing a structure that the gripping piece 723 is in a closed state with respect to the body section 721; FIG. 14(2) is an illustration showing a structure that the gripping piece 723 is in an open state with respect to the body section 721.

As shown in FIG. 14(1), the suction forceps 720 for endoscopic surgery includes a hollow section 722, and includes a gripping piece 723 formed pivotally with respect to the body section 721 around the pivot 721b as a pivot center, and a suction port 725 formed in a bottom surface portion 721d on a tip side of the body section 721. The suction port 725 is formed as a circular opening in communication with the hollow section 722 regardless the open state and the closed state of the gripping piece 723 with respect to the body section 721 as shown in FIG. 14(1), and FIG. 14(2).

As different from the embodiments described above, with the embodiment shown in FIG. 14, the suction port 725 is formed only in the bottom surface 721d of the body section 721 as separated independently from the gripping piece 723. For the suction forcepses 700, 710 for endoscopic surgery, it is required to manufacture the body sections and the gripping pieces receptively with accuracy of a certain degree so as to form the circular suction port in communication with the hollow section where the gripping piece is in the closed state with respect to the body section. To the contrary, in the embodiment shown in FIG. 14, the suction forceps can be manufactured with a lower accuracy than the suction forcepses 700, 710 because the suction port 725 is formed only in the bottom surface portion 721d of the body section 721. Therefore, according to the embodiment shown in FIG. 14. the suction forceps 720 for endoscopic surgery can be provided in being obtainable of substantially the same advantages as those of the suction forcepses 700, 710 while suppressing its manufacturing costs.

As described above, according to the suction forceps for endoscopic surgery in accordance with embodiments, liquids such as blood and body liquids can be suctioned quickly without any need of re-insertion of a particular suction pipe even where blood or body liquids are leaked out during the surgery, and consequently the surgery time can be prevented from taking longer.

1-16. (canceled)

17. A suction forceps for endoscopic surgery comprising:
a hollow section formed in a body section;
a gripping part formed on a tip of the body section on one end side of the body section for handling tissues during surgery; and
a suction port formed in communication with the hollow section for suctioning effused blood and body liquid leaking from the tissues during the surgery.

18. The suction forceps for endoscopic surgery according to claim 17, wherein the gripping part includes an annular portion fitting to an inclined end surface portion formed in an oval shape at the tip of the body section on one end side of the body section and functions as a blood clot crushing member for crushing the blood clot by clamping the blood clot with the annular portion and the inclined end surface portion.

19. The suction forceps for endoscopic surgery according to claim 18, wherein the inclined end surface portion is formed with an end surface side opening for forming the suction port, and the annular portion fitting the inclined end surface portion is formed with a crushing opening having a diameter smaller than that of the end surface side opening.

20. The suction forceps for endoscopic surgery according to claim 17, further comprising a reservoir section formed in communication with the hollow section at the other end side of the body section having the gripping part formed for reserving the blood and the body liquid suctioned through the suction port.

21. The suction forceps for endoscopic surgery according to claim 20, wherein the reservoir section has a body liquid absorbing material for absorbing the blood and the body liquid.

22. The suction forceps for endoscopic surgery according to claim 20, wherein the reservoir section has a connection pipe detachably attached to a suction pipe extending from an external suction device.

23. The suction forceps for endoscopic surgery according to claim 22, further comprising a switch for turning on and off suction operation using the external suction device while connected to the suction pipe from the external suction device via the connection pipe.

24. The suction forceps for endoscopic surgery according to claim 17, further comprising an illuminating means for externally illuminating light via the suction port.

25. The suction forceps for endoscopic surgery according to claim 17, further comprising a connection terminal connecting a conduction line extending from an external electric coagulation apparatus, wherein the suction forceps coagulates in a carbonizing manner a part at which bleeding is to be stopped based on a power generated at the external electric coagulation apparatus.

26. The suction forceps for endoscopic surgery according to claim 25, wherein the connection terminal is connected to the gripping part and is formed at an end of a manipulation wiring on an end side of thereof for manipulating the gripping part.

27. The suction forceps for endoscopic surgery according to claim 17, further comprising a suction stopping mechanism for stopping suction operation done at the suction port.

28. The suction forceps for endoscopic surgery according to claim 17, wherein the suction stopping mechanism is arranged at the gripping part.

29. The suction forceps for endoscopic surgery according to claim 28, wherein the gripping part includes a gripping piece portion arranged pivotally to the body section for performing suction and stopping suction at the suction port in accordance with pivotal movement of the gripping piece portion with respect to the body section.

30. The suction forceps for endoscopic surgery according to claim 27, wherein the suction port is formed on the tip of the body section on one end side of the body section.

31. The suction forceps for endoscopic surgery according to claim 27, wherein the suction port is formed on a side surface of the body section.

32. The suction forceps for endoscopic surgery according to claim 27, wherein the suction port is formed on a bottom surface of the body section.
